(19) United States
(12) Patent Application Publication
(10) Pub. No.: US 2009/0023683 A1
(43) Pub. Date: Jan. 22, 2009

(54) COMPLEXES COMPRISING ZOLEDRONIC ACID AND CYCLODEXTRINS

(76) Inventors: Chandrasekhar Kocherlakota, Secunderabad (IN); Paul Sudhakar Mogadati, Hyderabad (IN); Sarveswara Rao Mandavilli, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/173,871

(22) Filed: Jul. 16, 2008

Related U.S. Application Data
(60) Provisional application No. 60/949,991, filed on Jul. 16, 2007.

(51) Int. Cl. A61K 31/716; A61P 3/14

(52) U.S. Cl. 514/58

(57) ABSTRACT

The present invention relates to stable compositions comprising zoledronic acid, wherein zoledronic acid is complexed with a cyclodextrin, such as hydroxypropyl-β-cyclodextrin. The invention further relates to processes for making the compositions.

### INTRODUCTION TO THE INVENTION

The present invention relates to pharmaceutical formulations of zoledronic acid, including pharmaceutically acceptable salts, solvates, single isomers, enantiomers and mixtures thereof. More particularly, this invention relates to zoledronic acid complexed with cyclodextrins and pharmaceutical compositions comprising complexes of zoledronic acid and cyclodextrins for parenteral use.

Zoledronic acid has a chemical name (1-Hydroxy-2-(1H-imidazol-1-yl-ethylidene]-biphosphonic acid, or (1-Hydroxy-2-imidazol-1-yl-phosphonoethyl) phosphonic acid. It is useful in the treatment of hypercalcemia of malignancy, multiple myeloma and bone metastases of solid tumors and is commercially available in products sold using the trademark ZOMETA®, as a concentrate for infusion in vials containing 4.264 mg zoledronic acid monohydrate (equivalent to 4 mg of anhydrous zoledronic acid) per 5 ml, and as a powder for reconstitution having 4 mg of anhydrous zoledronic acid per vial. The products are manufactured by Boehringer Mannheim GmbH and marketed by Novartis. The structural formula for zoledronic acid monohydrate is Formula I.

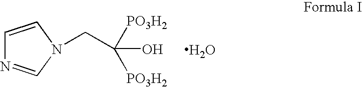

U.S. Pat. No. 4,777,163 describes processes for the preparation of alkyldiphosphonic acid derivatives and usefulness of the derivatives for the treatment or prophylaxis of calcium metabolism imbalance.

International Application Publication No. WO 01/52859, U.S. Patent Application Publication No. 2001/0011082 and U.S. Pat. No. 6,677,320 disclose a parenteral composition comprising a bisphosphonate and a pharmaceutically acceptable chelating agent, for parenteral administration.

International Application Publication No. WO 2005/025551 describes a heat-sterilisable plastic material container containing a bisphosphonate infusion solution.

Since zoledronic acid is sparingly water-soluble, its solubility limits its use in parenteral formulations, because of the requirement for large amounts of solvent. Hence there is an unmet need to increase aqueous solubility of zoledronic acid that will decrease the amount of solvent required for its parenteral administration. The present invention addresses this long-felt need of solubility enhancement of zoledronic acid by formation of a water-soluble complex with cyclodextrin.

### SUMMARY OF THE INVENTION

The present invention relates to pharmaceutical formulations comprising zoledronic acid, including pharmaceutically acceptable salts, solvates, single isomers, enantiomers and mixtures thereof.

An aspect of the present invention provides complexes comprising zoledronic acid and cyclodextrins.

In an embodiment, a cyclodextrin in said zoledronic acid and cyclodextrin complex comprises hydroxypropyl-β-cyclodextrin.

In an embodiment, the zoledronic acid and cyclodextrin complexes have molar ratios of zoledronic acid to cyclodextrin in the range of about 1:0.01 to about 1:10.

An aspect of the present invention provides aqueous solutions comprising zoledronic acid or its pharmaceutically acceptable salt and cyclodextrins, for parenteral administration.

In an embodiment, the pH of an aqueous solution comprising zoledronic acid and a cyclodextrin for parenteral administration ranges between about 3.5 and about 8.2.

In another embodiment, the pH of an aqueous solution comprising zoledronic acid and a cyclodextrin for parenteral administration ranges between about 4 and about 5.5.

### BRIEF DESCRIPTION OF THE DRAWINGS

FIG. 1 shows DSC thermogram curves related to a zoledronic acid and HPβCD complex of molar ratio 1:0.1, as prepared in Example 1. A, B, and C represent pure zoledronic acid, zoledronic acid and HPβCD complex, and placebo (prepared according to the example, but omitting the drug compound), respectively.

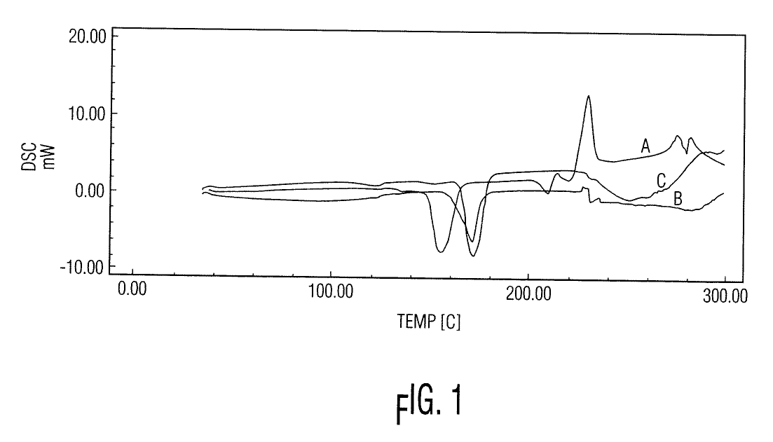

FIG. 2 shows DSC thermogram curves related to a zoledronic acid and HPβCD complex of molar ratio 1:0.25, as prepared in Example 2. A, B, and C represent pure zoledronic acid, zoledronic acid and HPβCD complex, and placebo, respectively.

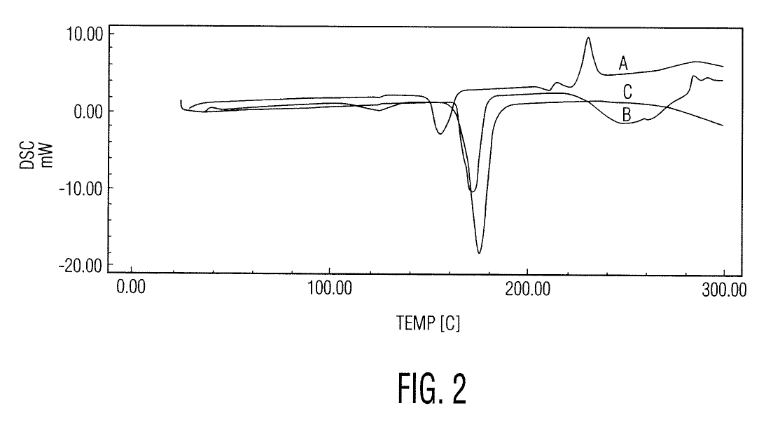

FIG. 3 shows DSC thermogram curves related to a zoledronic acid and HPβCD complex of molar ratio 1:0.5, as prepared in Example 3. A, B, and C represent pure zoledronic acid, zoledronic acid and HPβCD complex, and placebo, respectively.

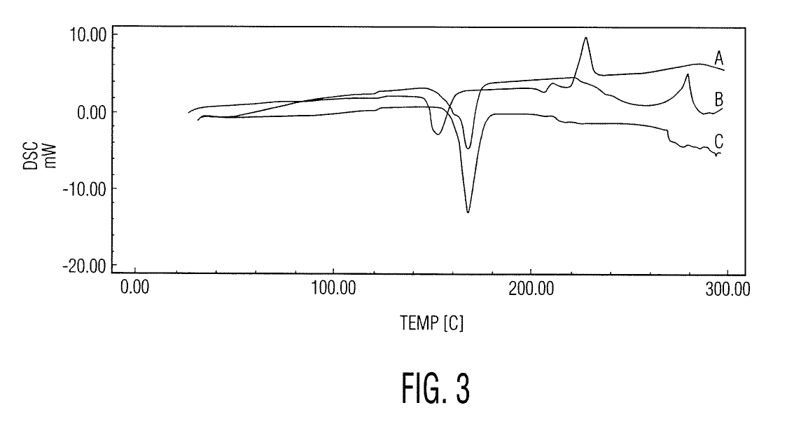

FIG. 4 shows DSC thermogram curves related to a zoledronic acid and HPβCD complex of molar ratio 1:1, as prepared in Example 4. A, B, and C represent pure zoledronic acid, zoledronic acid and HPβCD complex, and placebo, respectively.

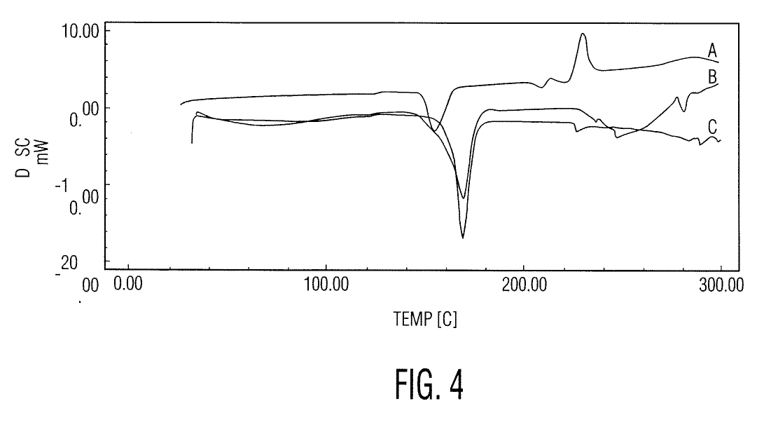

FIG. 5 shows DSC thermogram curves related to a zoledronic acid and HPβCD complex of molar ratio 1:0.5, as prepared in Example 5. A, B, and C represent pure zoledronic acid, zoledronic acid and HPβCD complex and placebo, respectively.

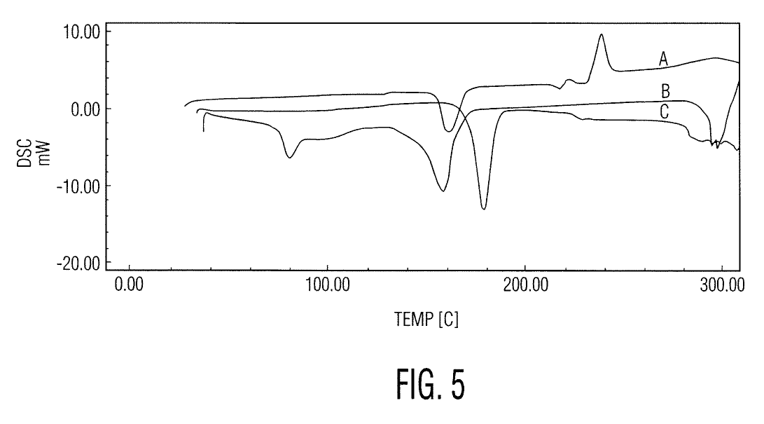

FIG. 6 is an XRD pattern of pure zoledronic acid.

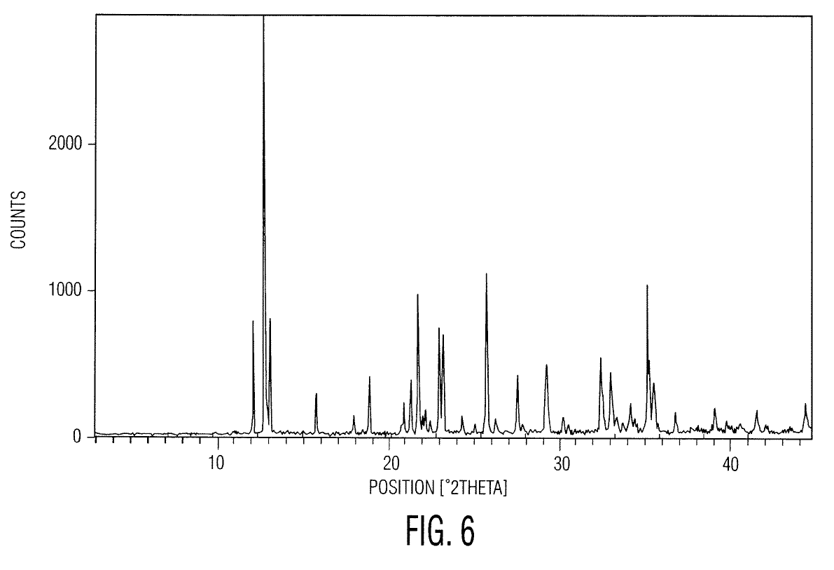

FIG. 7 is an XRD pattern of a zoledronic acid and HPβCD complex of molar ratio 1:0.1, as prepared in Example 1.

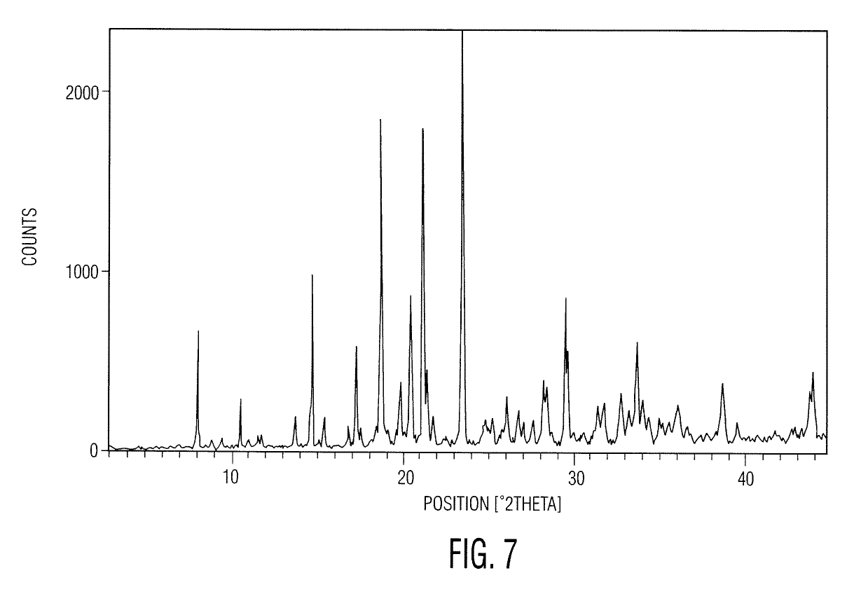

FIG. 8 is an XRD pattern of a zoledronic acid and HPβCD complex of molar ratio 1:0.25, as prepared in Example 2.

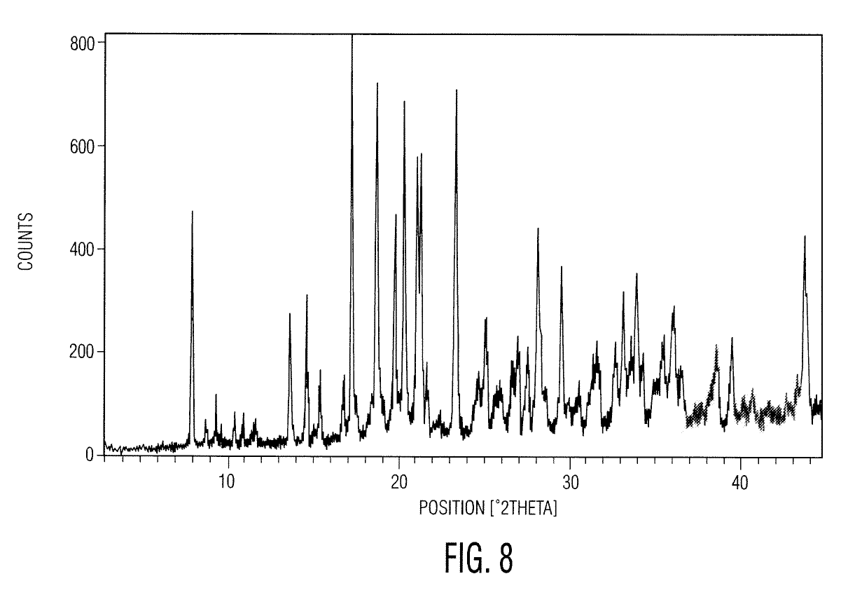

FIG. 9 is an XRD pattern of a zoledronic acid and HPβCD complex of molar ratio 1:0.5, as prepared in Example 3.

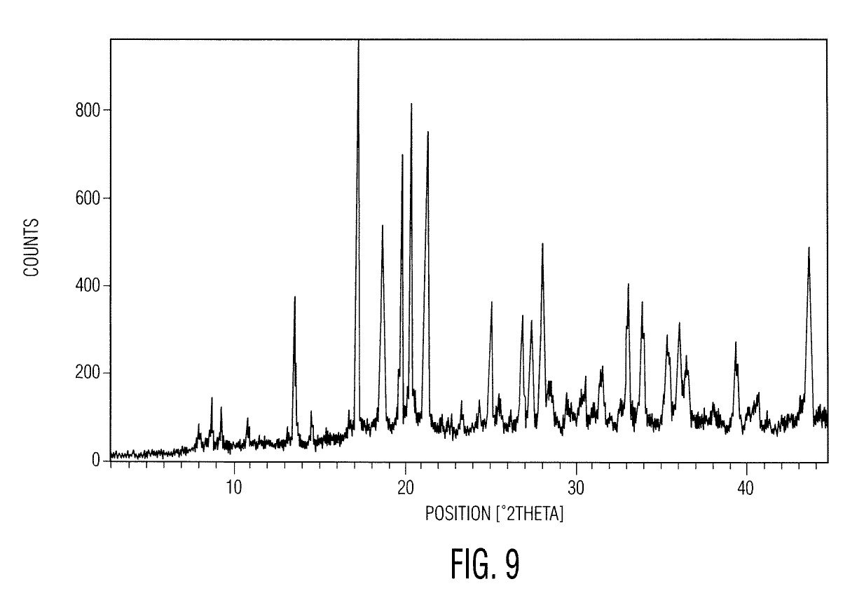

FIG. 10 is an XRD pattern of a zoledronic acid and HPβCD complex of molar ratio 1:1, as prepared in Example 4.

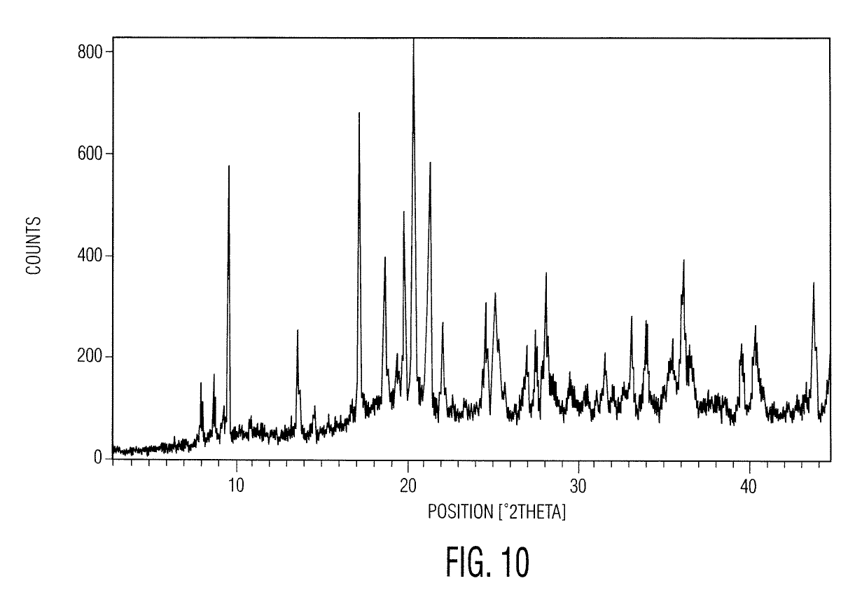

FIG. 11 is an XRD pattern of a zoledronic acid and HP βCD complex of molar ratio 1:0.5, as prepared in Example 5.

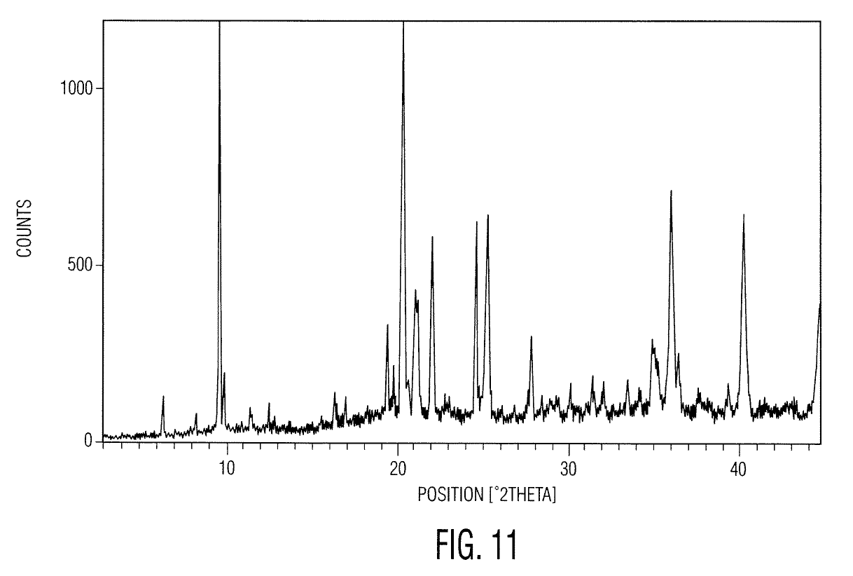

FIG. 12 is a FTIR spectrum of pure zoledronic acid.

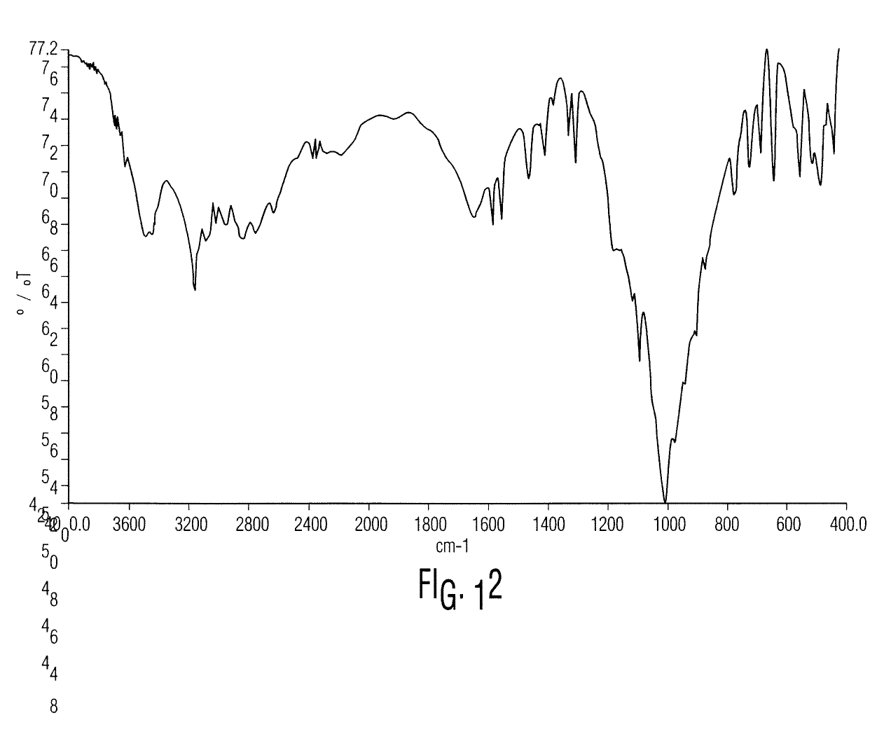

FIG. 13 is a FTIR spectrum of a zoledronic acid and HPβCD complex of molar ratio 1:0.1, as prepared in Example 1.

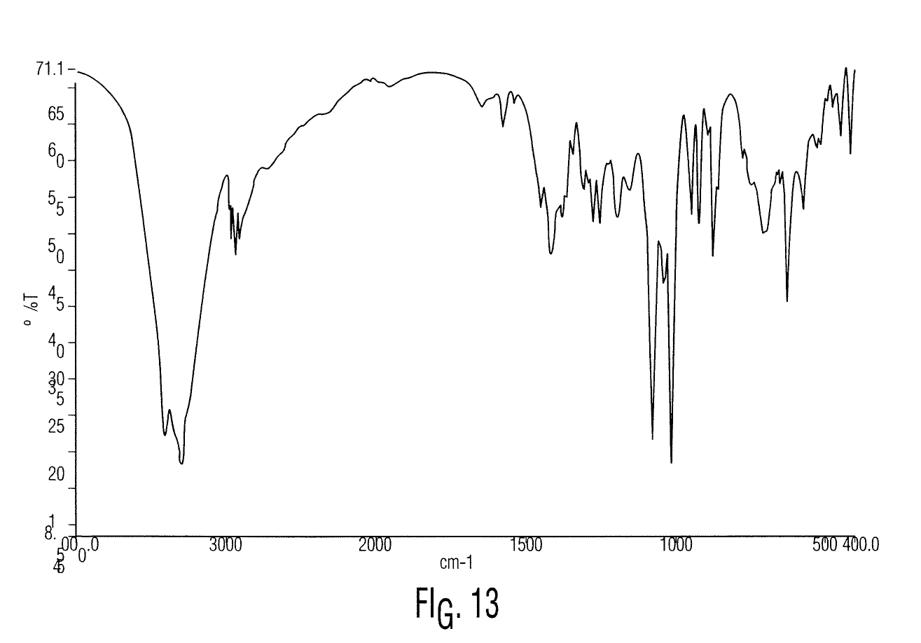

FIG. 14 is a FTIR spectrum of a zoledronic acid and HPβCD complex of molar ratio 1:0.25, as prepared in Example 2.

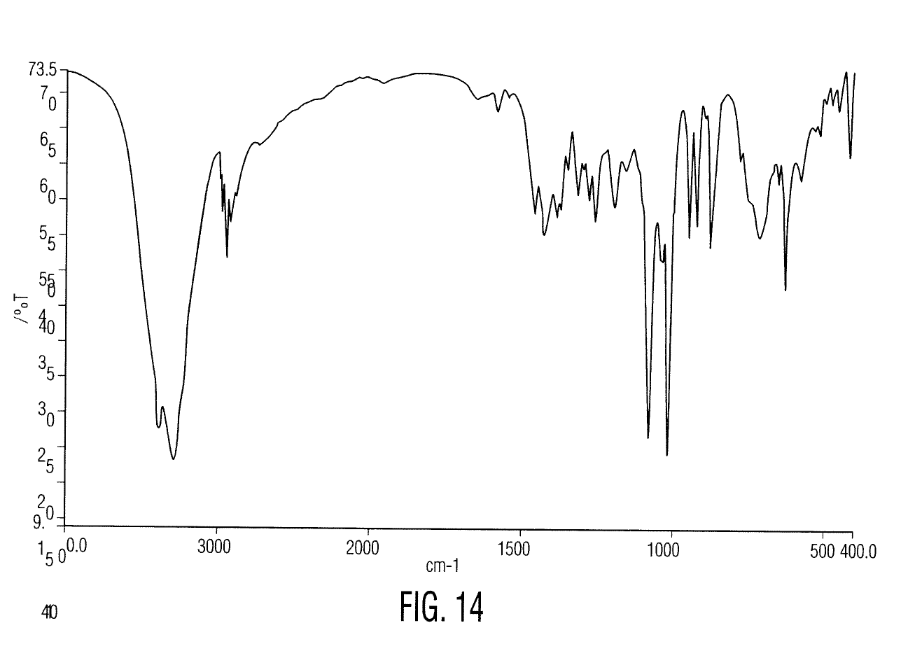

### DETAILED DESCRIPTION

The present invention relates to pharmaceutical formulations comprising zoledronic acid, including its pharmaceutically acceptable salts, hydrates, solvates, single isomers, enantiomers, and mixtures thereof.

Zoledronic acid, being sparingly water-soluble, does not give a clear solution at higher concentrations, hence providing challenges to the developmental pharmacist in developing suitable parenteral formulations. Due to this, the amount of zoledronic acid needed to be administered for clinically effective treatment and the volume of solvent necessary to be administered parenterally may be clinically unacceptable. Typically, the greater the volume needed to be administered parenterally to a patient, the longer the infusion time, the higher the likelihood of a vehicle-related adverse effect, the more expensive the product, and the less likelihood that the formulation will be found acceptable by the patient.

In an embodiment, a zoledronic acid used in the context of the present invention comprises zoledronic acid monohydrate.

In an embodiment, a zoledronic acid used in the context of the present invention comprises zoledronic acid trihydrate, disclosed in U.S. Patent Application Publication No. 2006/0178439.

A cyclodextrins (“CD”) is a cyclic oligosaccharide possessing hydrophobic cavities. CDs can be useful in combination with various drugs either for complexation or as auxiliaries such as diluents, solubilizers or tableting ingredients. An advantage of using CDs mainly comes from their inclusion complex formation. Complexation can protect a drug molecule and can eventually have considerable pharmaceutical potential.

There are various advantages for drug delivery using inclusion complex formation. Incompatible drugs can be mixed when one of them is complexed with a CD. The release rate of drugs can be controlled. The solubility of water-insoluble drugs can be improved. The instability of drugs in water and the acidic environment of the stomach conditions can be improved, since the rate of hydrolysis, photo-decomposition, auto-catalytic reactions, etc., are considerably reduced.

The present invention provides zoledronic acid complexes with cyclodextrins for parenteral use, rendering the complex water-soluble. Thus, the formulation so-obtained forms a clear solution, which is administrable parenterally.

The concentration of a cyclodextrin needed to effectuate solubilization depends on the type of solvent employed, the particular cyclodextrin utilized, and the conditions under which the solvent is maintained as well as the concentration of the drug in the solvent.

In the present invention, cyclodextrins of various grades that may be used include, but are not limited to: α-cyclodextrins, β-cyclodextrins (“β-CD”), and γ-cyclodextrins; derivatives of β-cyclodextrin such as hydroxypropyl-β-cyclodextrin (“HPβCD”), methylated β-cyclodextrin, maltosyl-β-cyclodextrin, sulphobutyl ether β-cyclodextrin, dimethyl-β-cyclodextrin, and the like; derivatives of α-cyclodextrin such as methylated α-cyclodextrin, hydroxyethyl-α-cyclodextrin, hydroxypropyl-α-cyclodextrin, and maltosyl-α-cyclodextrin; and derivatives of γ-cyclodextrin such as methylated γ-cyclodextrin, hydroxypropyl γ-cyclodextrin, maltosyl γ-cyclodextrin, and hydroxyethyl γ-cyclodextrin. Other substituted cyclodextrins and mixtures of more than one cyclodextrin and/or cyclodextrin derivatives may also be used and are within the scope of this invention. The term “cyclodextrin” as used herein includes the cyclodextrin compounds and their substituted derivatives.

βCD is a cyclic oligosaccharide consisting of seven glucose units. HPβCD is a βCD molecule having hydroxy groups substituted with hydroxypropyl groups, the basic closed circular structure of βCD being maintained in HPβCD. The glycosidic oxygen forming the bond between the adjacent glucose monomers and the hydrogen atoms lining the cavity of the cyclodextrin impart an electron density and hydrophpbic character to the cavity. Organic compounds interact with the walls of the cavity to form inclusion complexes. The hydroxyl groups and the hydroxypropyl groups are on the exterior of the molecule and interact with water to provide the increased aqueous solubility of the HPβCD and the complexes made with the HPβCD.

The hydroxypropyl groups are randomly substituted onto the hydroxyl groups of the cyclodextrin and the amount of substitution is reported as average degree of substitution, or number of hydroxypropyl groups per cyclodextrin, and is the preferred manner of describing the substitution. Substitution is a distribution around the average degree of substitution (the number of hydroxypropyl groups per cyclodextrin) with some molecules having more than the average and some less than the average degree of substitution. The result is a mixture of many molecular species with respect to the number and location of substitutions around the ring of the cyclodextrin.

Substitution can have an effect on the binding of guests to the HPβCD. At low degrees of substitution, binding is very similar to that of the unmodified β-cyclodextrin. Increasing substitution can lead to weakened binding due to steric hindrance. The effect is dependent upon the particular guest and it is also possible to obtain increased binding due to an increase in surface area to which the guest can bind. With most guests, these differences in binding with degree of substitution are small if detectable.

HPβCD has been used as a drug carrier due to its low toxicity, high tolerance and excellent solubilizing and stabilizing abilities. HPβCD has generally been found to be safe and no adverse effects are observed in human studies. HPβCD provides a good balance of enhanced aqueous solubility and of forming stable complexes. HPβCD is itself very soluble in water (greater than 500 mg/ml at room temperature, compared to 18 mg/ml for β-cyclodextrin). Additionally, β-cyclodextrin is considered to be nephrotoxic, so it is not recommended for use in parenteral formulations.

Several substitution degrees of HPβCD can be complexed with zoledronic acid, such as HPβCD with average substitution 5.32, HPβCD with average substitution 4.34, HPβCDs with average substitution 3.29, 4.7, 5.4, 6.7, and 9.8, and the like. A commercial source for HPβCD is Yiming Fine Chemicals Co., Ltd., Jiangsu, China, in various average hydroxypropyl contents.

Other commercially available cyclodextrins may be used such as those available from any of the commercial suppliers including: Cargill, Inc, Wayzata, Minn. USA; Roquette Freres, Lestrem, France; Aldrich Chemical Company, Milwaukee, Wis. USA;. and Wacker Chemicals, New Canaan, Conn. USA; or the cyclodextrins may be synthesized by various processes known in the art for the synthesis of cyclodextrins and their derivatives.

An aspect of the present invention provides complexes comprising zoledronic acid and a cyclodextrin.

In embodiments, the cyclodextrin in the zoledronic acid and cyclodextrin complexes comprises a β-cyclodextrin or a derivative thereof. HPβCD has been found to be useful for complex formation with zoledronic acid in the context of the present invention. The complexation is evident from the differential scanning calorimetry (“DSC”) curves, X-ray powder diffraction (“XRPD”) patterns, and Fourier-transform infrared (“FTIR”) absorption spectra obtained from the formulations of the present invention. XRD patterns described herein were obtained using copper Kα radiation. FTIR spectra were obtained from samples of the complexes compressed into potassium bromide pellets.

According to the present invention, molar ratios of zoledronic acid to cyclodextrin or substituted cyclodextrin in the zoledronic acid and cyclodextrin complexes range from about 1:0.01 to about 1 :10, or from about 1:0.1 to about 1:2.

Water-soluble sugars and sugar derivatives can be included as bulking agents in the formulation, such as but not limited to mannitol, mono-, di-, and poly-saccharides such as dextrose, lactose, and maltodextrin, and the like.

Another aspect of the present invention provides an aqueous solution comprising zoledronic acid or its pharmaceutically acceptable salt and a cyclodextrin, optionally with other pharmaceutically excipients, for parenteral administration.

In embodiments, the pH of the aqueous solution comprising zoledronic acid and cyclodextrin for parenteral administration ranges between about 3.5 to about 8.2. In certain embodiments, the pH of the aqueous solution comprising zoledronic acid and cyclodextrin for parenteral administration ranges between about 4 and about 5.5.

The present invention also includes the use of organic alkalizers or inorganic salts useful for pH adjustment such as, but not limited to, sodium or potassium carbonate, arginine, tromethamine, meglumine, sodium or potassium acetate, and the like.

Additionally, any polymer, sugar, polyhydric alcohol, salt, salt combination, aqueous solvent, mixed aqueous and non-aqueous solvents, and the like, may be employed as a solubilizing adjunct if the compound is biocompatible with desired product stability, as is known to a person skilled in the art.

Further an isotonising agent can also be used so as to make the formulation isotonic for parenteral use. The examples of such isotonising agents include, but not limited to, glycerol, polyethylene glycol, propylene glycol, ethanol, amino acids, sugars, sodium nitrate, potassium chloride, urea, ammonium chloride and the like.

The pharmaceutical formulations of the present invention may contain one or more excipients so that it becomes easier for the person skilled in the art to formulate and for the caregiver to handle.

An embodiment of the present invention provides unit doses of zoledronic acid ranging between 0.1 mg and 50 mg per vial, for parenteral administration.

In embodiments of the present invention, zoledronic acid complexed with cyclodextrin can also be administered orally using suitable solid oral dosage forms such as, but not limited to, tablets, capsules, caplets, powders for reconstitution, and the like. The pharmaceutical aids such as, but not limited to, fillers, diluents, binders, lubricants, and the like that are required to formulate such oral dosage forms are known to a person skilled in the art.

In an embodiment, the zoledronic acid complexed with cyclodextrin in accordance with the present invention is prepared by mixing an aqueous solution of HPβCD with zoledronic acid at temperatures ranging between about 5° C. and 95° C., or between about 20° C. and 75° C. The pH is adjusted using an agent such as sodium carbonate and a final clear solution is obtained and filtered using a suitable filter. Other processes for manufacturing pharmaceutical parenteral compositions known to a person skilled in the art fall within the scope of the present invention.

One embodiment of the present invention provides pharmaceutical formulations of zoledronic acid complexed with cyclodextrin for parenteral use, in solution form, or alternatively as a dry powder for reconstitution, prepared using suitable drying techniques such as but not limited to lyophilization, spray drying, tray drying, vacuum drying, fluidized bed drying, agitated thin-film drying, and the like.

Drying may be performed in a single step or in multiple steps with the conditions of drying differing between steps. Optionally, drying is performed under sterile or aseptic conditions. Optimum lyophilization conditions may vary based on the equipment design. As would be understood by one of ordinary skill in the art, many processes for drying the product in stable form may be employed in addition to freeze-drying.

The choice of the drying method will be determined by the composition to be dried and other considerations well known to a person skilled in the art.

Such dried powder may be reconstituted by mixing with a suitable liquid diluent such as, but not limited to, water for injection, normal saline solution, dextrose saline solution, and the like or mixtures thereof, before parenteral use. The compositions may be supplied as a kit comprising a container, such as a vial, having therein a dry powder comprising zoledronic acid, and another container having therein a reconstituting liquid. In an embodiment, the containers are separated chambers in a single device, such as a two-component syringe that permits mixing its contained components before injection.

In embodiments, the aqueous formulations comprising zoledronic acid and cyclodextrin are intended for parenteral administration. “Parenteral administration” used herein means intra-venous, intra-arterial, intra-peritoneal, intra-tumoral, subcutaneous administration, implantable injections, depots and the like.

In another embodiment, compositions of the present invention comprise pharmaceutically acceptable additives for parenteral use such as but not limited to stabilizers, antioxidants, pH modifiers and others, as known to a person skilled in the art.

Non-limiting examples of pH modifiers, buffers and stabilizers include citric acid, tartaric acid, succinic acid, glutamic acid, ascorbic acid, lactic acid, acetic acid, malic acid, maleic acid, and sodium salts thereof, sodium hydroxide, sodium carbonate, sodium bicarbonate, tris buffer, meglumine, amino acids and mixtures thereof. Such pH modifiers and stabilizers maintain a desired pH between about 2 and 10, or between about 2.5 and 5.5 in the composition.

The present invention may be part of a kit or device and be filled into vials, ampoules and any other form of packaging, which will allow ease of application parenterally.

The following examples will further describe certain specific aspects and embodiments of the invention, are provided solely for purposes of illustration, and are not to be construed as limiting the scope of the invention in any manner.

### EXAMPLES 1-3

### Preparation of Zoledronic Acid-HPβCD Complexes with Various Molar Ratios

| | Quantity | | |
|---|---|---|---|
| Ingredient | Example 1* | Example 2** | Example 3*** |
| Zoledronic acid | 6.25 g | 5.75 g | 5 g |
| monohydrate | | | |
| Hydroxypropyl-β- | 3.04 g | 6.99 g | 12.15 g |
| cyclodextrin | | | |
| Sodium carbonate | q.s. to pH 3.5 | q.s. to pH 3.5 | q.s. to pH 3.5 |
| Mannitol | 31.25 g | 28.75 g | 25 g |

*Zoledronic acid to HPβCD molar ratio 1:0.1.
**Zoledronic acid to HPβCD molar ratio 1:0.25.
***Zoledronic acid to HPβCD molar ratio 1:0.5.

### EXAMPLES 4-5

### Preparation of Zoledronic Acid-HPβCD Complexes with Various Molar Ratios

Quantity

| Ingredient | Example 4* | Example 5** |
|---|---|---|
| Zoledronic acid | 3.75 g | 5 g |
| monohydrate | | |
| Hydroxypropyl-β- | 18.23 g | 12.15 g |
| cyclodextrin | | |
| Sodium carbonate | q.s. to pH 3.5 | q.s. to pH 8.2 |
| Mannitol | 18.75 g | 25 g |

*Zoledronic acid to HPβCD molar ratio 1:1.
**Zoledronic acid to HPβCD molar ratio 1:0.5.

### EXAMPLE 6

### Preparation of Zoledronic Acid Trihydrate-HPβCD Complex (Molar Ratio 1:1)

| Ingredient | Quantity |
|---|---|
| Zoledronic acid trihydrate | 2.1 g |
| Hydroxypropyl-β-cyclodextrin | 9.1 g |
| Sodium carbonate | q.s. to pH 3.5 |
| Mannitol | 9.4 g |

Manufacturing Process for Examples 1-6:

1. 150 mL of water was heated to 50-60° C.

2. Weighed quantity of HPβCD was added to hot water and stirred until a clear solution was obtained.

3. Weighed quantity of zoledronic acid was added to the solution and further stirred at 50-60° C. for 15 minutes.

4. pH was adjusted to 3-4, except in Example 5 where pH was adjusted to 8.2, with 1 M sodium carbonate and stirred until a clear solution was obtained.

5. Weighed quantity of mannitol was added to the solution with stirring until a clear solution was obtained

6. The solution was filtered through a 0.45 μm filter.

7. Filtrate was concentrated in a Buchi Rotavapor at 50-60° C. under vacuum to remove water until no droplet formation in the condenser was observed.

8. The wet mass obtained from concentration was collected by scraping the inner walls of the Rotavapor flask and dried on filter paper at 50° C. for 2 hours in a hot air oven.

### EXAMPLES 7-8

### Composition for Zoledronic Acid Trihydrate Capsule Formulation

| | mg/Capsule | |
|---|---|---|
| Ingredient | EXAMPLE 7 | EXAMPLE 8 |
| Zoledronic acid trihydrate-HPβCD | 8 | 40 |
| complex (Example 6) | | |
| Mannitol | 91 | 59 |
| Magnesium stearate | 1 | 1 |
| Total | 100 | 100 |

Manufacturing Process:

1. Zoledronic acid trihydrate-HPβCD complex obtained from Example 6 and mannitol were passed through an ASTM #40 mesh sieve and mixed together in a blender to attain uniformity.

2. Magnesium stearate was passed through an ASTM #60 mesh sieve.

3. The step 2 magnesium stearate was mixed with step 1 zoledronic acid blend in a blender to attain uniformity.

4. The blend was filled in size 2 capsules using a capsule filling machine.

### EXAMPLE 9

### Solubility data of Examples 1-5 and Zoledronic Acid Monohydrate

| Zoledronic acid monohydrate to | |
|---|---|
| Hydroxypropyl-β- | Water Solubility at |
| cyclodextrin Molar Ratio | 25° C. (mg/mL) |
| 1:0.1 (Example 1) | 15 |
| 1:0.25 (Example 2) | 35 |
| 1:0.5 (Example 3) | 80 |
| 1:1 (Example 4) | 150 |
| 1:0.5 (Example 5) | 80 |
| Zoledronic acid monohydrate | <7 |

### EXAMPLE 10

### Composition and Stability of Zoledronic Acid Injection Formulation

| Ingredient | Quantity | |
|---|---|---|
| Zoledronic acid monohydrate | 864.2 | mg |
| Hydroxypropyl-β-cyclodextrin | 433 | mg |
| Mannitol | 44 | g |
| Sodium carbonate | q.s. to pH 4-4.5 | |
| Water for injection | q.s. to 1000 | mL |

Manufacturing Process:

1. About 750 mL water for injection at 50-60° C. was used to dissolve HPβCD.

2. Weighed quantity of zoledronic acid was added to the solution and further stirred to dissolve.

3. pH of the solution was adjusted using 1 M sodium carbonate solution.

4. Mannitol was added to the solution and dissolved with stirring.

5. Volume of the solution was made up to 1000 mL with water for injection.

6. The solution was sterilized by filtration through a 0.22 μm nylon filter.

7. Filtrate was filled into sterile vials aseptically.

8. The vials were closed using sterile ETFE-coated butyl stoppers, and sealed.

Samples were tested for stability by storage under various temperature and relative humidity (“RH”) conditions with periodic analyses, and the data are given below, where HPLC impurity concentrations and the drug assay are expressed as percentages of the label drug concentration.

| | | | Drug | | Unknown | Total |
|---|---|---|---|---|---|---|
| Storage | Physical | | Assay | IAA Impurity* | Impurity | Impurities |
| Conditions | Description | pH | (% w/w) | (%) | (%) | (%) |
| Initial | Clear, | 4.87 | 100.7 | ND** | ND | ND |
| | colorless | | | | | |
| | solution | | | | | |
| 1 Month at | Clear, | 4.81 | 101.2 | ND | ND | ND |
| 40° C. and | colorless | | | | | |
| 75% RH | solution | | | | | |
| 2 Months at | Clear, | 4.68 | 101.2 | ND | 0.01 | 0.01 |
| 40° C. and | colorless | | | | | |
| 75% RH | solution | | | | | |
| 6 Months at | Clear, | 4.98 | 102.5 | 0.01 | ND | 0.01 |
| 40° C. and | colorless | | | | | |
| 75% RH | solution | | | | | |
| 6 Months at | Clear, | 4.98 | 101.2 | ND | ND | ND |
| 30° C. and | colorless | | | | | |
| 60% RH | solution | | | | | |
| 6 Months at | Clear, | 4.97 | 100 | ND | ND | ND |
| 25° C. and | colorless | | | | | |
| 60% RH | solution | | | | | |

*Imidazoleacetic acid, a zoledronic acid degradation product.
**ND: Not detected.

### EXAMPLE 11

### Composition of Zoledronic Acid Trihydrate Injection Formulation

| Ingredient | Quantity | |
|---|---|---|
| Zoledronic acid trihydrate | 960.4 | mg |
| Hydroxypropyl-β-cyclodextrin | 428.1 | mg |
| Mannitol | 44 | g |
| Sodium carbonate | q.s. to pH 4-4.5 | |
| Water for injection | q.s. to 1000 | mL |

Manufacturing process was similar to that described in Example 10.

We claim:

1. A complex comprising zoledronic acid and a cyclodextrin.

2. The complex of claim 1, wherein a cyclodextrin is a hydroxypropyl-β-cyclodextrin.

3. The complex of claim 1, wherein a molar ratio of zoledronic acid to cyclodextrin is about 1:0.01 to about 1:10.

4. The complex of claim 1, wherein a molar ratio of zoledronic acid to cyclodextrin is about 1:0.1 to about 1:2.

5. A pharmaceutical formulation comprising zoledronic acid and a cyclodextrin.

6. The pharmaceutical formulation of claim 5, comprising a complex of zoledronic acid and a cyclodextrin.

7. The pharmaceutical formulation of claim 6, wherein a cyclodextrin is a hydroxypropyl-β-cyclodextrin.

8. The pharmaceutical formulation of claim 6, wherein a molar ratio of zoledronic acid to cyclodextrin in a complex is about 1:0.1 to about 1:2.

9. The pharmaceutical formulation of claim 6, in the form of a parenteral composition.

10. The pharmaceutical formulation of claim 6, further comprising a water-soluble sugar or sugar derivative.

11. The pharmaceutical formulation of claim 6, being a solid for reconstitution with a liquid diluent.

12. The pharmaceutical formulation of claim 6, being a solid composition for oral administration.

13. A pharmaceutical formulation comprising a lyophilized powder containing a complex of zoledronic acid and a hydroxypropyl-β-cyclodextrin, wherein the complex has a molar ratio of zoledronic acid to hydroxypropyl-β-cyclodextrin about 1:0.1 to about 1:2.

14. The pharmaceutical formulation of claim 13, further comprising a water-soluble sugar or sugar derivative.

15. The pharmaceutical formulation of claim 13, further comprising a pH modifier.

16. The pharmaceutical formulation of claim 15, wherein a pH modifier provides a pH about 3.5 to about 8.2 in a liquid injectable composition formed from the lyophilized powder.

17. The pharmaceutical formulation of claim 13, further comprising a water-soluble sugar or sugar derivative, and a pH modifier.

18. A kit for treating a hypercalcemia condition, comprising a container having therein a pharmaceutical formulation of claim 13 and a container having therein a liquid diluent.
